(19) United States
(12) Patent Application Publication
(10) Pub. No.: US 2016/0067180 A1
(43) Pub. Date: Mar. 10, 2016

(54) SEMI-SOLID CHEWABLE DOSAGE FORM FOR OVER-THE-COUNTER MEDICATIONS AND METHOD FOR PRODUCING SAME

(71) Applicant: SANTA CRUZ PHARMACEUTICALS, INC., Santa Cruz, CA (US)

(72) Inventors: Michael T. WESTHUSING, Alamo, CA (US); Yong BAI, Santa Cruz, CA (US); Mario W. MEDRI, Miami Lakes, FL (US)

(73) Assignee: SANTA CRUZ PHARMACEUTICALS, INC., Santa Cruz, CA (US)

(21) Appl. No.: 14/848,043

(22) Filed: Sep. 8, 2015

Related U.S. Application Data
(63) Continuation-in-part of application No. 14/626,897, filed on Feb. 19, 2015.
(60) Provisional application No. 62/115,618, filed on Feb. 12, 2015.
(60) Provisional application No. 62/046,712, filed on Sep. 5, 2014.

(51) Int. Cl. A61K 9/00; A61K 31/137; A61K 31/4402; A61K 31/194; A61K 31/09; A61K 31/485; A61K 31/695; A61K 33/10; A61K 31/426; A61K 31/4439; A61K 31/138; A61K 47/36; A61K 47/26; A61K 47/10; A61K 47/12; A61K 31/438

(52) U.S. Cl. CPC A61K 9/0056; A61K 31/438; A61K 31/137; A61K 31/4402; A61K 31/194; A61K 31/09; A61K 31/485; A61K 31/695; A61K 33/10; A61K 31/426; A61K 31/4439; A61K 31/138; A61K 47/36; A61K 47/26; A61K 47/10; A61K 47/12

(57) ABSTRACT

The invention provides a semi-solid chewable dosage form for use as an over-the-counter medication that contains an active pharmaceutical ingredient to treat symptoms associated with allergies, colds, coughs, fever, pain, gastrointestinal disorders, sleep, and other common conditions. The invention further provides a semi-solid chewable dosage form that contains an active pharmaceutical ingredient, a gelling agent, gelatin, sugar, a polyol, and a pH adjusting agent. The invention also provides a semi-solid chewable dosage form that contains a gelling agent, sugar, a polyol, glycerin, and a pH adjusting agent.

### CROSS-REFERENCE TO RELATED APPLICATIONS

This patent application claims the benefit of U.S. Provisional Application No. 62/115,618, filed Feb. 12, 2015, and is a continuation-in-part of U.S. patent application Ser. No. 14/626,897, filed Feb. 19, 2015, which claims the benefit of U.S. Provisional Application No. 62/046,712, filed Sep. 5, 2014, which are incorporated herein by reference in their entireties for all purposes.

### BACKGROUND OF THE INVENTION

Over-the-counter (OTC) medications are commonly used to treat various symptoms associated with allergies, colds, coughs, fever, pain, gastrointestinal disorders, and sleep. OTC medications are available in a variety of solid dosage forms that are taken orally including tablets, capsules, and soft-gels.

The oral administration of solid dosage forms is difficult for some individuals who have difficulties swallowing any type of pills. This problem is magnified for solid dosage forms that need to be taken 2-4 times per day to provide the desired therapeutic effect. In addition, solid dosage forms often have an unpleasant after-taste. Moreover, the need for a source of water or other liquid to assist with swallowing solid dosage forms can complicate administration. All of these problems with traditional solid oral dosage forms lead to sub-optimal patient compliance with taking OTC medications resulting in the patient suffering symptoms for extended periods of time.

Liquid suspensions or solutions are sometimes used as an alternative to solid oral dosage forms. However, the dosing with liquid dosage forms is not precise which can lead to the administration of too little or too much medications. In addition, liquid dosage forms are messy and often have a bitter taste which leads to problems with patient compliance.

The need remains for alternative dosage forms for OTC medications to treat symptoms associated with allergies, colds, coughs, pain, gastrointestinal disorders and other common conditions that are suitable for oral administration without an unpleasant taste or problem with swallowing to improve patient compliance.

### BRIEF SUMMARY OF THE INVENTION

An embodiment of the invention provides a semi-solid chewable dosage form that contains an active pharmaceutical ingredient, a gelling agent, gelatin, sugar, a polyol, and a pH adjusting agent.

In another embodiment, the invention provides a semi-solid chewable dosage form that contains an active pharmaceutical ingredient, a gelling agent, gelatin, sugar, corn syrup, and a pH adjusting agent.

A further embodiment of the invention provides a semi-solid chewable dosage form that contains an active pharmaceutical ingredient, a gelling agent, gelatin, sugar and corn syrup. In another embodiment, the invention provides a semi-solid chewable dosage form that contains an active pharmaceutical ingredient, a gelling agent, gelatin, sugar, and a polyol.

The active pharmaceutical ingredient may be chlorpheniramine maleate, phenylephrine hydrochloride, guaifenesin, dextromethorphan hydrobromide, loratadine, diphenhydramine, or a combination thereof. Alternatively, the active pharmaceutical ingredient useful for treating gastrointestinal disorders may be an antacid, an anti-foaming agent, a histamine H2-receptor antagonist (commonly known as a H2 antagonist), proton pump inhibitor, or a combination thereof. Other active pharmaceutical ingredients commonly present in OTC medications are suitable for use in the present invention.

A method of producing a semi-solid chewable dosage form is provided. The method comprises forming a primary blend comprising a gelling agent, sugar, a polyol and a pH adjusting agent, cooking the primary blend to obtain a residual moisture content to between 5% by weight to 25% by weight, combining the primary blend with a secondary blend containing an active pharmaceutical ingredient to yield a final blend, depositing the final blend into individual semi-solid chewable dosage forms.

In a further embodiment, the invention provides a method of producing a semi-solid chewable dosage form that comprises forming a primary blend comprising a gelling agent, sugar, and corn syrup, cooking the primary blend to obtain a residual moisture content to between 5% by weight to 25% by weight, combining the primary blend with a secondary blend containing an active pharmaceutical ingredient to yield a final blend, depositing the final blend into individual semi-solid chewable dosage forms.

In another embodiment, the invention provides a method of producing a semi-solid chewable dosage form that comprises forming a primary blend comprising a gelling agent, sugar, and a polyol, cooking the primary blend to obtain a residual moisture content to between 5% by weight to 25% by weight, combining the primary blend with a secondary blend containing an active pharmaceutical ingredient to yield a final blend, depositing the final blend into individual semi-solid chewable dosage forms.

The semi-solid chewable dosage form according to the invention is useful for administration to individuals, including both adults and children, to treat symptoms from allergies, colds, coughs, pains, fever, gastrointestinal disorders, sleep, and the like.

Other embodiments, characteristics, and advantages of the invention are apparent after reading the descriptions and examples that follow.

### DETAILED DESCRIPTION OF THE INVENTION

The semi-solid chewable dosage form of the invention (also referred to as the semi-solid dosage form) is intended to be chewed by a patient such that it is broken up into smaller parts within the oral cavity and then easily swallowed. The semi-solid dosage form has a sufficiently high viscosity that it is not pourable and further does not flow or conform to its container at room temperature. Typically, the semi-solid dosage form does not flow at low shear stress and generally exhibits plastic flow behavior. In general, the consistency of the semi-solid dosage form is the same as or similar to gelatin-based or pectin-based candy products such as, for example, gummy bears and pectin jellies.

The dosage form can have any size and shape such that it can be administered orally and chewed by a patient. The patient should be able to readily break apart the dosage form by chewing and swallow the dosage form without the need for an external source of liquid. Typically, the dosage form has a length of about 1 cm to about 5 cm, width of about 1 cm to about 5 cm and a height of about 1 cm to about 5 cm. Suitable shapes include, for example, ovals, spheres, cylinders, rectangular boxes and cubes. The dosage form may be formed into unique shapes and figures including, for example, animals for administration to children (e.g., under the age of 13) and/or adults.

Generally, each individual dosage form has a total weight of at least 100 mg. Typically, each dosage form has a total weight of from about 1 g to about 20 g. Preferably, each dosage form has a total weight of from about 1 g to about 15 g. Preferably each dosage form has a total weight of from about 1 g to about 10 g, for example, about 1 g to about 1.5 g, about 1.5 g to about 2 g, about 2 g to about 2.5 g, about 2.5 g to about 3 g, about 3.5 g to about 4 g, about 4 g to about 4.5 g, about 4.5 g to about 5 g, about 5 g to about 5.5 g, about 5.5 g to about 6 g, about 6 g to about 6.5 g, about 6.5 g to about 7 g, about 7 g to about 7.5 g, about 7.5 g to about 8 g, about 8 g to about 8.5 g, about 8.5 g to about 9 g, about 9 g to about 9.5 g, and about 9.5 g to about 10 g. Most preferably, each dosage form has a total weight of about 5 g.

Active pharmaceutical ingredients that are suitable for use in the semi-solid dosage form for the invention include, by way of example, anti-allergy, antihistamines, antitussives, decongestants, expectorants, anti-cold/flu, analgesics, anti-inflammatories, sleep medications, anti-heartburn medications, anti-gas medications, anti-GERD medications, anti-diarrheals, laxatives, anti-smoking and/or motion sickness medications. In some embodiments, suitable active pharmaceutical ingredients may treat and/or prevent gastrointestinal disorders including, for example, antacids, anti-foaming agents, H2 antagonists, proton pump inhibitors, anti-diarrheals, laxatives, or a combination thereof. Suitable active pharmaceutical ingredients useful in the semi-solid dosage form of the invention are typically available as over-the-counter medications.

The semi-solid chewable dosage of the invention is further useful for administration to individuals, including both adults and children, to treat and/or prevent allergies, colds and coughs, as well as symptoms of these conditions. Additionally, the semi-solid chewable dosage form of the invention may be used to treat or prevent gastrointestinal disorders and symptoms thereof such as dyspepsia, peptic ulcer, gastroesophageal reflux disease, upset stomach, heartburn, excessive gas, and the like along with symptoms of these disorders.

In other embodiments, the semi-solid chewable dosage form of the invention includes one or more active pharmaceutical ingredients useful for the treatment of gastrointestinal disorders and symptoms thereof. Such active ingredients are typically available as over-the-counter medications.

Any suitable active pharmaceutical ingredients may be used in the semi-solid dosage form of the present invention to treat or prevent one or more symptoms. Suitable active pharmaceutical ingredients are set forth in Table 1:

TABLE 1

Anti-Allergy
loratadine
diphenhydramine
cetirizine
fexofenadine
Anti-Cold/Flu
pseudoephedrine
phenylephrine
chlorpheniramine
Analgesics
ibuprofen
aspirin
naproxen
acetaminophen
codeine
Cough Medications
guaifenesin
dextromethorphan
Sleep Medications
diphenhydramine
doxylamine
Heartburn Medications
ranitidine
cimetidine
famotidine
omeprazole
esomeprazole
lansoprazole
calcium carbonate
bismuth subsalicylate
Anti-Diarrheals
loperamide
Anti-Gas
simethicone
Laxatives
bisacodyl
Smoking Cessation
nicotine
Motion Sickness
dimenhydrinate
meclazine

Combinations of two or more active pharmaceutical ingredients may be used in the semi-solid dosage form of the invention to treat or prevent one or more symptoms. Suitable combinations of active pharmaceutical ingredients are set forth in Table 2:

TABLE 2

Anti-Allergy
loratadine/pseudoephedrine
diphenhydramine/phenylephrine, diphenhydramine/pseudoephedrine
cetirizine/pseudoephedrine
fexofenadine/pseudoephedrine
Anti-Cold/Flu
chlorpheniramine/phenylephrine
ibuprofen/phenylephrine
dextromethorphan/ibuprofen/phenylephrine
dextromethorphan/acetaminophen/phenylephrine
acetaminophen/phenylephrine, acetaminophen/pseudoephedrine
doxylamine/dextromethorphan/acetaminophen
acetaminophen/dextromethorphan/guifenesin/phenylephrine
Analgesics
ibuprofen/caffeine
aspirin/caffeine
naproxen/caffeine
acetaminophen/caffeine
diphenhydramine/ibuprofen
diphenhydramine/acetaminophen
diphenhydramine/naproxen
acetaminophen/caffeine/aspirin
Antitussives
guaifenesin/dextromethorphan
Sleep Medications
diphenhydramine/ibuprofen
diphenhydramine/aspirin
diphenhydramine/naproxen
diphenhydramine/acetaminophen
Anti-Gas
simethicone/calcium carbonate

Pharmaceutically acceptable salts of any suitable active pharmaceutical ingredients may also be used.

Preferably, the active pharmaceutical ingredients used in the semi-solid chewable dosage form of the invention include chlorpheniramine maleate, phenylephrine hydrochloride, guaifenesin, dextromethorphan hydrobromide, loratadine, diphenhydramine, or a combination thereof. In one embodiment, the dosage form contains a combination of chlorpheniramine maleate and phenylephrine hydrochloride. In another embodiment, the dosage form contains a combination of dextromethorphan hydrobromide and phenylephrine hydrochloride.

Chlorpheniramine maleate is a pharmaceutically acceptable salt of chlorpheniramine. Chlorpheniramine has the following chemical structure:

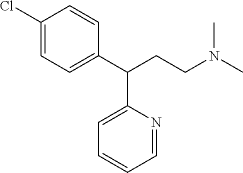 [SMILES: CN(C)CCC(c1ccc(Cl)cc1)c1ccccn1]

In some embodiments, the amount of chlorpheniramine maleate present in each dosage form is from about 0.1 mg to about 30 mg. Preferably, the amount of chlorpheniramine maleate present in each dosage form is from about 1 mg to about 10 mg. More preferably, the amount of chlorpheniramine maleate present in each dosage form is about 1 mg to about 5 mg. Most preferably, the amount of chlorpheniramine present is about 2 mg or about 4 mg in each dosage form that has a total weight of about 5 g.

Alternatively, chlorpheniramine maleate may be present in the dosage form in amount from about 0.01% by weight to about 1.0% by weight, and preferably about 0.02% to about 0.2% by weight. For an adult dose, chlorpheniramine maleate is preferably present in an amount from about 0.06% by weight to about 0.1% by weight. For a pediatric dose (e.g., children under 13), chlorpheniramine maleate is preferably present in an amount from about 0.03% by weight to about 0.05% by weight.

Phenylephrine hydrochloride is a pharmaceutically acceptable salt of phenylephrine. Phenylephrine has the following chemical structure:

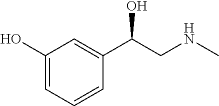

In some embodiments, the amount of phenylephrine hydrochloride present in each dosage form is from about 0.1 mg to about 20 mg. Preferably, the amount of phenylephrine hydrochloride present is from about 2 mg to about 15 mg. More preferably, the amount of phenylephrine hydrochloride present is from about 3 mg to about 12 mg. Most preferably, the amount of phenylephrine hydrochloride present is about 5 mg or about 10 mg in each dosage form that has a total weight of about 5 g.

Alternatively, phenylephrine hydrochloride may be present in the dosage form in amount from about 0.01% by weight to about 1% by weight, and preferably 0.01% to about 0.5% by weight. For an adult dose, phenylephrine hydrochloride is preferably present in an amount from about 0.15% by weight to about 0.25% by weight. For a pediatric dose (e.g., children under 13), phenylephrine hydrochloride is preferably present in an amount from about 0.05% by weight to about 0.15% by weight.

Guaifenesin has the following chemical structure:

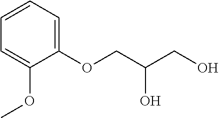

In some embodiments, the amount of guaifenesin present in each dosage form is from about 10 mg to about 1,500 mg. Preferably, the amount of guaifenesin present in each dosage form is from about 200 mg to about 1,200 mg. More preferably, the amount of guaifenesin present in each dosage form is about 100 mg to about 400 mg. Most preferably, the amount of guaifenesin present is about 100 mg, 200 mg or about 400 mg in each dosage form that has a total weight of about 5 g.

Alternatively, guaifenesin may be present in the dosage form in amount from about 0.1% by weight to about 20% by weight, and preferably about 0.5% to about 10% by weight. For an adult dose, guaifenesin is preferably present in an amount from about 0.5% by weight to about 5% by weight. For a pediatric dose (e.g., children under 13), guaifenesin is preferably present in an amount from about 0.1% by weight to about 4% by weight.

Dextromethorphan hydrobromide is a pharmaceutically acceptable salt of dextromethorphan. Dextromethorphan has the following chemical structure:

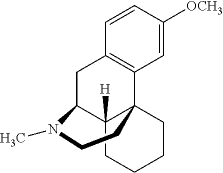

In some embodiments, the amount of dextromethorphan hydrobromide present in each dosage form is from about 1 mg to about 100 mg. Preferably, the amount of guaifenesin present in each dosage form is from about 5 mg to about 60 mg. More preferably, the amount of guaifenesin present in each dosage form is about 10 mg to about 30 mg. Most preferably, the amount of guaifenesin present is about 10 mg or about 20 mg in each dosage form that has a total weight of about 5 g.

Alternatively, dextromethorphan hydrobromide may be present in the dosage form in amount from about 0.01% by weight to about 2% by weight, and preferably about 0.1% to about 1% by weight. For an adult dose, guaifenesin is preferably present in an amount from about 0.1% by weight to about 1% by weight. For a pediatric dose (e.g., children under 13), guaifenesin is preferably present in an amount from about 0.1% by weight to about 0.8% by weight.

Loratadine has the following chemical structure:

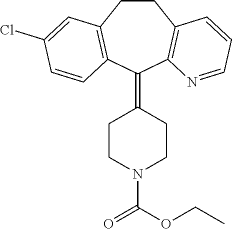 [SMILES: CCOC(=O)N1CCC(=C2c3ccc(Cl)cc3CCc3cccnc32)CC1]

In some embodiments, the amount of loratadine present in each dosage form is from 1 mg to about 100 mg. Preferably, the amount of loratadine present in each dosage form is from about 5 mg to about 50 mg. More preferably, the amount of loratadine present in each dosage form is from about 10 mg to about 30 mg. Most preferably, the amount of loratadine present is about 10 mg in each dosage form that has a total weight of about 5 g.

Alternatively, loratadine may be present in the dosage form in amount from about 0.01% by weight to about 2% by weight, and preferably about 0.1% to about 1% by weight. For an adult dose, loratadine is preferably present in an amount from about 0.1% by weight to about 1% by weight. For a pediatric dose (e.g., children under 13), loratadine is preferably present in an amount from about 0.1% by weight to about 0.5% by weight.

Diphenhydramine hydrochloride is a pharmaceutically acceptable salt of diphenhydramine. Diphenhydramine has the following chemical structure:

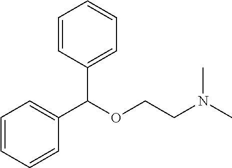 [SMILES: CN(C)CCOC(c1ccccc1)c1ccccc1]

In some embodiments, the amount of diphenhydramine hydrochloride present in each dosage form is from 1 mg to about 100 mg. Preferably, the amount of diphenhydramine hydrochloride present in each dosage form is from about 5 mg to about 50 mg. More preferably, the amount of diphenhydramine hydrochloride present in each dosage form is from about 10 mg to about 30 mg. Most preferably, the amount of diphenhydramine hydrochloride present is about 12.5 mg or 25 mg.

Alternatively, diphenhydramine hydrochloride may be present in the dosage form in amount from about 0.01% by weight to about 2% by weight, and preferably about 0.1% to about 1% by weight. For an adult dose, diphenhydramine hydrochloride is preferably present in an amount from about 0.1% by weight to about 1% by weight. For a pediatric dose (e.g., children under 13), diphenhydramine hydrochloride is preferably present in an amount from about 0.1% by weight to about 0.5% by weight.

Chlorpheniramine maleate and phenylephrine hydrochloride may optionally both be present in combination in the dosage form. Preferably, in such embodiments, chlorpheniramine maleate is present in an amount of about 2 mg and phenylephrine hydrochloride is present in an amount of about 5 mg. Alternatively, chlorpheniramine maleate is present in an amount of about 4 mg and phenylephrine hydrochloride is present in an amount of about 10 mg. Typically, a pediatric dose contains about 2 mg chlorpheniramine maleate and 5 mg phenylephrine hydrochloride and an adult dose contains about 4 mg chlorpheniramine maleate and 10 mg phenylephrine hydrochloride.

In embodiments in which dextromethorphan hydrobromide and phenylephrine hydrochloride are both present in the dosage form, preferably dextromethorphan hydrobromide is present in an amount of about 10 mg and phenylephrine hydrochloride is present in an amount of about 5 mg. Alternatively, dextromethorphan hydrobromide is present in an amount of about 20 mg and phenylephrine hydrochloride is present in an amount of about 10 mg.

In other embodiments, the active pharmaceutical ingredient is an antacid, anti-foaming agent, histamine H2-antagonist, proton pump inhibitor, anti-diarrheal, laxative, or combination thereof, that are useful for the treatment and/or prevention of gastrointestinal disorders or symptoms thereof.

Suitable antacids including, but are not limited to, potassium bicarbonate, sodium bicarbonate, calcium bicarbonate, aluminum bicarbonate, magnesium bicarbonate, magnesium hydroxide, calcium carbonate, aluminum hydroxide, and combinations thereof.

In a preferred embodiment, the antacid is calcium carbonate. Calcium carbonate has the formula Ca₂CO₃. The calcium carbonate can be anhydrous calcium carbonate or a hydrate thereof.

Suitable histamine H2-receptor antagonists include, but are not limited to, cimetidine, ranitidine, famotidine, and nizatidine.

In a preferred embodiment, the histamine H2-receptor antagonist is famotidine. Famotidine has the structure:

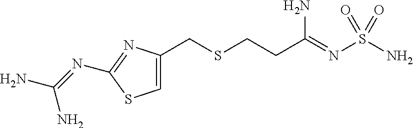 [SMILES: NC(N)=Nc1nc(CSCC/C(N)=N/S(N)(=O)=O)cs1]

Suitable proton pump inhibitors include, but are not limited to, omeprazole, lansoprazole, dexlansoprazole, esomeprazole, pantoprazole, rabeprazole, and ilaprazole.

In a preferred embodiment, the proton pump inhibitor is omeprazole. Omeprazole has the structure:

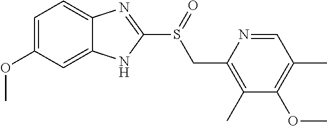 [SMILES: COc1ccc2nc(S(=O)Cc3ncc(C)c(OC)c3C)[nH]c2c1]

A suitable anti-diarrheal includes, but is not limited to, loperamide.

A suitable laxative includes, but is not limited to, bisacodyl.

A suitable anti-gas agent includes, but is not limited to, simethicone. Simethicone has the following structure:

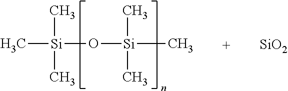

The amount of active pharmaceutical ingredient in the semi-solid dosage form will vary for each different active depending on its use. The amount present will usually be less for semi-solid dosage forms that are intended to be administered to children. For semi-solid dosage forms for use with gastrointestinal disorders, the active ingredient may be present in an amount sufficient to treat and/or prevent gastrointestinal disorders and symptoms thereof (e.g., bloating, discomfort and/or pain) including, for example, dyspepsia, peptic ulcer, gastroesophageal reflux disease, upset stomach, heartburn, and excessive gas.

Typically, the semi-solid dosage form contains about 0.1 mg to 1 g of the active pharmaceutical ingredient. Alternatively, the semi-solid dosage form contains one or more active pharmaceutical ingredients in an amount from about 0.01% by weight to about 10% by weight.

The semi-solid dosage form of the invention may be administered once per day or multiple times per day to provide relief for various symptoms affecting an individual. For example, chlorpheniramine maleate may be administered to treat symptoms of allergic rhinitis or sinusitis. Phenylephrine hydrochloride may be administered to treat symptoms of nasal congestion. Typical dosing of chlorpheniramine maleate for adults is 4 mg every 4-6 hours and for children (i.e., 6-11 years old) is 2 mg every 4-6 hours. Typical dosing of phenylephrine hydrochloride for adults is 10 mg every 4-6 hours and for children (i.e., 6-11 years old) is 5 mg every 4-6 hours.

Guaifenesin may be administered to treat symptoms of congestion in the chest and throat. Typical dosing of guaifenesin for adults is 200 mg to 400 mg every 4-6 hours and for children (i.e., 6-11 years old) is 100 mg to 200 mg every 4-6 hours.

Dextromethorphan hydrobromide may be administered to treat symptoms of a cough. Typical dosing of dextromethorphan hydrobromide for adults is 10 mg to 30 mg every 4-8 hours and for children (i.e., 6-11 years old) is 5 mg to 10 mg every 4 hours.

Loratadine may be administered to treat symptoms of allergic rhinitis and urticaria. Typical dosing of loratadine for adults and children (i.e., 6-11 years old) is 10 mg per day.

In embodiments where the active pharmaceutical ingredient is an antacid, the amount of antacid present in each dosage form is from about 10 mg to about 2 g. Preferably, the amount of antacid present in each dosage form is from about 100 mg to about 1 g. More preferably, the amount of antacid present in each dosage form is about 500 mg to about 1 mg. Most preferably, the amount of antacid present is about 750 mg or about 800 mg in each dosage form that has a total weight of about 5 g.

Alternatively, the antacid may be present in the dosage form in amount from about 1% by weight to about 30% by weight, and preferably about 5% to about 20% by weight.

In embodiments where the active pharmaceutical ingredient is an anti-foaming agent (also referred to herein as an anti-gas agent), the amount of anti-foaming agent present in each dosage form is from about 1 mg to about 500 mg. Preferably, the amount of anti-foaming agent present is from about 5 mg to about 250 mg. More preferably, the amount of anti-foaming agent present is from about 10 mg to about 100 mg. Most preferably, the amount of anti-foaming agent present is about 20 mg or about 80 mg in each dosage form that has a total weight of about 5 g.

Alternatively, the anti-foaming agent may be present in the dosage form in amount from about 0.01% by weight to about 5% by weight, and preferably 0.1% to about 5% by weight. For an adult dose, the anti-foaming agent is preferably present in an amount from about 0.15% by weight to about 0.25% by weight. For a pediatric dose (e.g., children under 13), the anti-foaming agent is preferably present in an amount from about 0.05% by weight to about 0.15% by weight.

In some embodiments, the active pharmaceutical ingredient is a histamine H2-receptor antagonist. In these embodiments, the amount of histamine H2-receptor antagonist present in each dosage form is from about 1 mg to about 500 mg. Preferably, the amount of histamine H2-receptor antagonist is from about 5 mg to about 250 mg. More preferably, the amount of histamine H2-receptor antagonist present is from about 10 mg to about 100 mg. Most preferably, the amount of histamine H2-receptor antagonist present is about 20 mg or about 80 mg in each dosage form that has a total weight of about 5 g.

In some embodiments, the active pharmaceutical ingredient is a proton pump inhibitor. In these embodiments, the amount of proton pump inhibitor present in each dosage form is from about 1 mg to about 500 mg. Preferably, the amount of proton pump inhibitor is from about 5 mg to about 250 mg. More preferably, the amount of proton pump inhibitor present is from about 10 mg to about 100 mg. Most preferably, the amount of proton pump inhibitor present is about 20 mg or about 80 mg in each dosage form that has a total weight of about 5 g.

The semi-solid dosage form of the invention includes a gelling agent. Any suitable gelling agent may be used to provide the dosage form with the desired characteristics including, for example, semi-solid structure, shape and texture. The gelling agent is typically a USP (U.S. Pharmacopeia) grade gelling agent. Preferably, the gelling agent is pectin.

Pectin is a purified carbohydrate obtained by aqueous extraction from citrus peel or apple pomace. Any suitable type of pectin may be use in the dosage form including, for example, high-methoxy pectin and low-methoxy pectin and combinations thereof. Low-methoxy pectin may be amidated which is often referred to as LMA pectin. Examples of suitable pectins are Genu® citrus pectin USP/100 and Genu® citrus pectin USP/200 from CP Kelco.

Pectin may be generally present in the semi-solid dosage form in an amount of from about 0.01% by weight to about 10% by weight. Preferably, pectin is present in an amount of from about 0.5% by weight to about 7% by weight, for example from about 0.5% to about 1%, from about 1% to about 1.5%, from about 1.5% to about 2%, from about 2% to about 2.5%, from about 2.5% to about 3%, from about 3% to about 3.5%, from about 3.5% to about 4%, from about 4% to about 4.5%, from about 4.5% to about 5%, from about 5% to about 5.5%, from about 5.5% to about 6%, from about 6% to about 6.5%, and from about 6.5% to about 7%. More preferably, pectin is present in an amount from about 1% by weight to about 5% by weight.

In some embodiments, the semi-solid dosage form of the invention includes gelatin. Without being bound by any theory, it is believed that the presence of gelatin assists with gelling of the semi-solid dosage form and further serves to mask the taste of the active ingredients.

Any suitable type of gelatin may be present in the dosage form. For example, the gelatin may be animal-derived gelatin, chemically-modified gelatin, physically-modified gelatin, and combinations thereof. Animal-derived gelatin may be derived from any suitable source such as, for example, pigskin or bovine bone.

Alternatively, the gelatin may be hydrolyzed gelatin. Hydrolyzed gelatin is also commonly known as hydrolyzed collagen, collagen hydrolysate, and collagen peptide. Hydrolyzed gelatin having a molecular weight ranging from about 2,500 to about 5,000 may be used. An example of a suitable hydrolyzed gelatin is Peptiplus® powder from Gelita.

Gelatin may be generally present in the semi-solid dosage form in an amount from about 0.01% by weight to about 15% by weight. Preferably, gelatin is present in an amount of from about 0.5% by weight to about 8% by weight, for example from about 0.5% to about 1%, from about 1% to about 1.5%, from about 1.5% to about 2%, from about 2% to about 2.5%, from about 2.5% to about 3%, from about 3% to about 3.5%, from about 3.5% to about 4%, from about 4% to about 4.5%, from about 4.5% to about 5%, from about 5% to about 5.5%, from about 5.5% to about 6%, from about 6% to about 6.5%, from about 6.5% to about 7%, from about 7% to about 7.5%, and from about 7.5% to about 8%. More preferably, gelatin is present in an amount from about 1% by weight to about 5% by weight.

The semi-solid dosage form of the invention includes sugar. Generally, sugar is present in an amount from about 30% by weight to about 99% by weight of the dosage form. Preferably, sugar is present in an amount from about 40% by weight to about 95% by weight, for example, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, and from about 90% to about 85%.

In some embodiments of the invention, the semi-solid dosage form includes a polyol. Polyols are also referred to as sugar alcohols. Without being bound by any theory, the presence of a polyol is believed to promote the stability of the semi-solid dosage form of the invention.

Suitable polyols include, for example, hydrogenated starch hydrolysates, isomalt, lactitol, maltitol, mannitol, sorbitol, erythritol, and xylitol. Combinations of polyols may be used. Preferably, the polyol is hydrolyzed starch hydrolysates (HSH). HSH typically contains substantial quantities of hydrogenated oligo- and poly-saccharides in addition to monomeric and dimeric polyols. HSH is commonly known to include polyglycitol. An example of a commercially available HSH is Hystar® 3375 syrup (75% solids), Hystar® 4075 and Hystar® 6075 supplied by SPI Polyols. Other commercially available HSH include 75/400 from Roquette and Stabilite® liquid HSH and Stabilite® powdered HSH supplied by Corn Products Specialty Ingredients.

One or more polyols may be present in the semi-solid dosage form in an amount from about 30% by weight to about 99% by weight. Preferably, one or more polyols may be present in an amount from about 40% by weight to about 90% by weight, for example, about 40% to about 50%, about 50% to about 60%, about 60% to about 70%, about 70% to about 80%, and about 80% to about 90%. Alternatively, one or more polyols may be present in an amount from about 40% by weight to about 60% by weight.

In embodiments in which one or more polyols are present, the ratio of polyol to sugar is typically from about 1:10 to about 10:1 by dry weight. Preferably, the ratio of polyol to sugar is from about 1:2 to about 2:1 by dry weight, for example, from about 1:1.5 to about 1:5.1.

In other embodiments, the ratio of polyol to gelling agent is from about 40:1 to about 1:1 by dry weight. Preferably, the ratio of polyol to gelling agent is from about 30:1 to about 10:1 by dry weight.

In some embodiments, the semi-solid dose form includes corn syrup. Corn syrup may be present without a polyol. Alternatively, corn syrup may be present in addition to a polyol. Any suitable corn syrup may be used, for example, corn syrup having 36-65 DE (dextrose equivalents), preferably corn syrup 42-43 DE. Corn syrup may contain about 50% by weight to about 90% by weight solids, preferably about 80% solids.

Corn syrup may be present in the semi-solid dosage form in an amount from about 30% by weight to about 99% by weight. Preferably, corn syrup may be present in an amount from about 40% by weight to about 90% by weight, for example, about 40% to about 50%, about 50% to about 60%, about 60% to about 70%, about 70% to about 80%, and about 80% to about 90%.

In embodiments in which corn syrup is present, the ratio of corn syrup to sugar is typically from about 1:10 to about 10:1 by dry weight. Preferably the ratio of corn syrup to sugar is from about 1:2 to about 2:1 by dry weight, for example, from about 1:1.5 to about 1:5.1.

In other embodiments, the ratio of corn syrup to gelling agent is from about 20:1 to about 1:1 by dry weight. Preferably, the ratio of corn syrup to gelling agent is from about 10:1 to about 2:1 by dry weight.

The semi-solid dosage form may optionally include a pH adjusting agent. Any suitable pH adjusting agent may be used that is sufficient to adjust the pH during the manufacture of the dosage form to yield the desired pH. By way of example, the pH adjusting agent may be sodium citrate, citric acid, sodium ascorbate and ascorbic acid. Two or more pH adjusting agents may be used. The pH adjusting agent may be supplied in solid form (e.g., as a powder) or in aqueous solution. For example, citric acid may be supplied in a 50% solution. Preferably, the pH adjusting agent is sodium citrate or citric acid. More preferably, both sodium citrate and citric acid are included in the semi-solid dosage form as pH adjusting agents.

The pH adjusting agent may be present in the semi-solid dosage form in an amount from about 0.1% by weight to about 5% by weight. Preferably, the pH adjusting agent may be present in an amount from about 1% to about 5% by weight, for example, from about 1% to about 1.5%, from about 1.5% to about 2%, from about 2% to about 2.5%, from about 2.5% to about 3%, from about 3% to about 3.5%, from about 3.5% to about 4.0%, from about 4% to about 4.5%, and from about 4.5% to about 5%.

In some embodiments, sodium citrate is present in an amount from about 0.1% by weight to about 1% by weight. Preferably, sodium citrate is present in an amount from about 0.1% by weight to about 0.5% by weight, for example, from about 0.1% to about 0.2%, from about 0.2% to about 0.3%, from about 0.3% to about 0.4%, and from about 0.4% to about 0.5%.

In other embodiments, citric acid is present (as 50% aqueous solution) in an amount from about 0.5% by weight to about 3% by weight, for example from about 0.5% to about 1%, from about 1% to about 1.5%, from about 1.5% to about 2%, from about 2% to about 2.5%, and from about 2.5% to about 3%.

In certain embodiments, the semi-solid dosage form contains glycerin, also commonly known as glycerol. Without being bound by any theory, glycerin is believed to function as an emollient to stability the dosage form during its preparation. Preferably, glycerin USP is used. In some embodiments, glycerin is present in the semi-solid dosage form in addition to the absence of gelatin.

Glycerin may be present in the semi-solid dosage form in an amount from about 0.1% by weight to about 10% by weight. Preferably, glycerin is present in an amount from about 0.5% by weight to about 5% by weight, for example from about 0.5% to about 1%, from about 1% to about 1.5%, from about 1.5% to about 2.0%, from about 2.0% to about 2.5%, from about 2.5% to about 3.0%, from about 3.0% to about 3.5%, from about 3.5% to about 4.0%, from about 4.0% to about 4.5%, and from about 4.5% to about 5.0%.

In some embodiments, the semi-solid dosage form contains a flavorant. Any suitable food-grade flavorant may be used to suppress the bitterness of the active ingredients to provide a pleasant taste to the dosage form upon chewing and swallowing. A mixture of two or more flavorants may be used to yield the desired taste characteristic.

Suitable flavorants include artificial sweeteners such as, for example, sucralose, acesulfame potassium, stevia, sodium saccharine, erythritol, and aspartame. Another suitable flavorant may be a fraction of the lactone group such as, for example, decalactone and dodecalactone (e.g., gamma dodecalactone). Lactone fractions are typically supplied in a propylene glycol solution, in particular from 0.5% to 1% in propylene glycol solution. The flavorant may be orange or cherry flavors. Alternatively, the flavorant may be menthol.

Preferably, the flavorant is an artificial sweetener. More preferably, the artificial sweetener is sucralose.

The flavorant may be present in an amount up to about 1% by weight, preferably up to about 0.5% by weight, for example, up to about 0.01%, up to about 0.05%, up to about 0.1%, up to about 0.2%, up to about 0.3%, up to about 0.4%, and up to about 0.5%. In certain embodiments, the amount of flavorant present is in a range bounded by any of the foregoing values. Fractions of the lactone group may be present in an amount of from about 1 ppm to 50 ppm, preferably from about 2 ppm to about 10 ppm, and more preferably from about 3 ppm to about 9 ppm.

A colorant may optionally be added to provide a suitable appearance for the semi-solid dosage form. Examples of suitable colorants include red or yellow dyes such as FD&C Red #40 and FD&C Yellow #6. Two or more colorants may be combined.

The semi-solid chewable dosage form of the invention generally has a water content, also referred to as a residual moisture content, of less than about 15% by weight, e.g., about 14% or less, about 13% or less, about 12% or less, about 11% or less, about 10% or less, about 9% or less, about 8% or less, about 7% or less, about 6% or less, or about 5% or less. In other embodiments, the water content of the semi-solid dosage form is in a range bounded by any of the foregoing values. Preferably, the water content of the semi-solid dosage form is from about 8% by weight to about 15% by weight.

In some embodiments, the semi-solid chewable dosage form comprises one or more active pharmaceutical ingredients, a gelling agent, gelatin, sugar, a polyol, and a pH adjusting agent. In other embodiments, the semi-solid chewable dosage form comprises one or more active pharmaceutical ingredients, a gelling agent, gelatin, sugar, corn syrup, and a pH adjusting agent. In other embodiments, the semi-solid chewable dosage form comprises one or more active pharmaceutical ingredients, a gelling agent, sugar, a polyol, glycerin, and a pH adjusting agent.

In some embodiments, the semi-solid chewable dosage form comprises one or more active pharmaceutical ingredients, pectin, sugar, hydrolyzed starch hydrolysate, hydrolyzed gelatin, and a pH adjusting agent. In other embodiments, the semi-solid chewable dosage form comprises one or more active pharmaceutical ingredients, pectin, sugar, corn syrup, hydrolyzed gelatin, and a pH adjusting agent.

In some embodiments, the semi-solid chewable dosage form comprises one or more active pharmaceutical ingredients, pectin, sugar, hydrolyzed starch hydrolysate, glycerin, and a pH adjusting agent.

In some embodiments, the semi-solid chewable dosage form comprises:

one or more active pharmaceutical ingredients in an amount from about 0.01% by weight to about 10% by weight;

pectin in an amount from about 0.5% by weight to about 7% by weight;

sugar in an amount from about 40% by weight to about 95% by weight;

hydrolyzed starch hydrolysate in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

In some embodiments, the semi-solid chewable dosage form comprises:

one or more active pharmaceutical ingredients in an amount from about 0.01% by weight to about 10% by weight;

pectin in an amount from about 0.5% by weight to about 7% by weight;

sugar in an amount from about 40% by weight to about 95% by weight;

corn syrup in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

In some embodiments, the semi-solid chewable dosage form comprises:

one or more active pharmaceutical ingredients in an amount from about 0.01% by weight to about 10% by weight;

pectin in an amount from about 0.5% by weight to about 7% by weight;

sugar in an amount from about 40% by weight to about 95% by weight;

hydrolyzed starch hydrolysate in an amount from about 40% by weight to about 90% by weight;

glycerin in an amount from about 0.1% by weight to about 5% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

In some embodiments, the semi-solid chewable dosage form comprises:

chlorpheniramine maleate;

phenylephrine hydrochloride;

pectin in an amount from about 0.5% by weight to about 7% by weight;

sugar in an amount from about 40% by weight to about 95% by weight;

hydrolyzed starch hydrolysate in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

In some embodiments, the semi-solid chewable dosage form comprises:

chlorpheniramine maleate;

phenylephrine hydrochloride;

pectin in an amount from about 0.5% by weight to about 7% by weight;

sugar in an amount from about 40% by weight to about 95% by weight;

corn syrup in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

In some embodiments, the semi-solid chewable dosage form comprises:

diphenhydramine hydochloride;

pectin in an amount from about 0.5% by weight to about 7% by weight;

sugar in an amount from about 40% by weight to about 95% by weight;

hydrolyzed starch hydrolysate in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

In some embodiments, the semi-solid chewable dosage form comprises:

loratadine;

pectin in an amount from about 0.5% by weight to about 7% by weight;

sugar in an amount from about 40% by weight to about 95% by weight;

hydrolyzed starch hydrolysate in an amount from about 40% by weight to about 90% by weight;

glycerin in an amount from about 0.1% by weight to about 5% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

In some embodiments, the semi-solid chewable dosage form comprises:

an active pharmaceutical ingredient selected from the group consisting of an antacid, an anti-foaming agent, a histamine H2-receptor antagonist, a proton pump inhibitor, or a combination thereof in an amount from about 0.01% by weight to about 10% by weight;

pectin in an amount from about 0.5% by weight to about 7% by weight;

sugar in an amount from about 40% by weight to about 95% by weight;

hydrolyzed starch hydrolysate in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight,

wherein the water content of the semi-solid dosage form is from about 8% by weight to about 15% by weight.

In some embodiments, the semi-solid chewable dosage form comprises:

an active pharmaceutical ingredient selected from the group consisting of an antacid, an anti-foaming agent, a histamine H2-receptor antagonist, a proton pump inhibitor, or a combination thereof in an amount from about 0.01% by weight to about 10% by weight;

pectin in an amount from about 0.5% by weight to about 7% by weight;

sugar in an amount from about 40% by weight to about 95% by weight;

corn syrup in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight,

wherein the water content of the semi-solid dosage form is from about 8% by weight to about 15% by weight.

In some embodiments, the semi-solid chewable dosage form comprises:

calcium carbonate, simethicone, famotidine, or omeprazole;

pectin in an amount from about 0.5% by weight to about 7% by weight;

sugar in an amount from about 40% by weight to about 95% by weight;

hydrolyzed starch hydrolysate in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight,

wherein the water content of the semi-solid dosage form is from about 8% by weight to about 15% by weight.

In some embodiments, the semi-solid chewable dosage form comprises:

calcium carbonate, simethicone, famotidine, or omeprazole;

pectin in an amount from about 0.5% by weight to about 7% by weight;

sugar in an amount from about 40% by weight to about 95% by weight;

corn syrup in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight,

wherein the water content of the semi-solid dosage form is from about 8% by weight to about 15% by weight.

The semi-solid chewable dosage form of the invention can be prepared by any suitable method including, for example, a batch process or a continuous process. In some embodiments, the components of the dosage form are first combined together in a suitable vessel. The components can be combined in any suitable order.

During manufacturing, water is typically added to the combination of some or all of the components to form a mixture that is the base for the semi-solid dosage form. In some embodiments, pectin, sugar, a polyol, and a pH adjusting agent are combined with water to form the base. Alternatively, pectin, sugar, corn syrup, and a pH adjusting agent are combined with water to form the base. Any amount of water may be added to prepare a suitable mixture. In some embodiments, a sufficient amount of water is added to dissolve water-soluble components, for example, sugar, and uniformly disperse non-water-soluble components to form a mixture.

Following the preparation of the base containing the components of the semi-solid dosage form along with water, the base typically has a water content of from about 10% by weight to about 90% by weight. Preferably, the base has a water content of from about 20% by weight to about 50% by weight, for example, about 20% to about 25%, about 25% to about 30%, about 30% to about 35%, about 35% to about 40%, about 40% to about 45%, and about 45% to about 50%.

In some embodiments, the base is cooked at a suitable temperature to remove a portion of the water present. By reducing the water content through cooking, the base may be converted into a semi-solid chewable dosage form having the desired physical characteristics, in particular consistency and texture. The base may be cooked by any suitable means including, for example, with a steam-jacketed vessel or a conventional heat exchanger. Cooking may optionally be carried out with the aid of a vacuum.

The base may be cooked at any suitable temperature and for a sufficient length of time to yield a molten mass having the desired water content. Generally, following cooking, the base has a residual moisture content from about 5% by weight to about 25% by weight. Preferably, the base has a residual moisture content after cooking from about 9% by weight to about 20% by weight, for example, about 9% to about 10%, about 10% to about 11%, about 11% to about 12%, about 12% to about 13%, about 13% to about 14%, and about 14% to about 15%, about 15% to about 16%, about 16% to about 17%, about 17% to about 18%, about 18% to about 19%, and about 19% to about 20%. In certain aspects, the residual moisture content of the base after cooking is an amount to provide a semi-solid dosage form containing about 0.01% by weight to about 2% by weight of the active ingredients.

Generally, the base is cooked at a temperature of from about 220° F. to about 265° F. Preferably, the base may be cooked at a temperature of about 230° F. to about 250° F., for example, about 230° F. to about 235° F., about 235° F. to about 240° F., about 240° F. to about 245° F., and about 245° F. to about 250° F.

After the base is cooked for a sufficient time to yield a molten mass, any remaining components of the semi-solid dosage form may be added such as, for example, the active pharmaceutical ingredients chlorpheniramine maleate and phenylephrine hydrochloride, hydrolyzed gelatin, glycerin, a flavorant, and a colorant to form the final blend. These additional components may be added to the base by any suitable means using, for example, mass flow meters and static mixers.

A pH adjusting agent, such as citric acid, may be added to the base to provide a suitable pH for the final blend that contains all of the components of the semi-solid dosage form. The pH of the final blend is generally from about 4 to about 6, preferably from about 4.5 to about 5.5.

In some embodiments, different blends of components are prepared separately and then combined together to form a final blend from which the semi-solid dosage form is obtained. For example, a primary blend may be combined with a secondary blend to form the final blend. A separate blend containing flavorants and/or colorants and an acid solution may optionally be added in the preparation of the final blend.

In one embodiment, a primary blend is prepared by combining pectin, sugar, a polyol, and a pH adjusting agent with water. Alternatively, the primary blend may be prepared by combining pectin, sugar, corn syrup, and a pH adjusting agent with water. The amount of water and corn syrup. A pH adjusting agent such as, for example, sodium citrate may optionally be added to the primary blend. In some embodiments, the primary blend has a pH from about 2 to about 6, preferably from about 2.5 to about 4, and more preferably from about 2.8 to about 3.8.

In certain aspects, the primary blend is cooked at an appropriate temperature and for an appropriate length of time to provide the primary blend with any suitable moisture content for further processing. Preferably, the primary blend has a moisture content after cooking from about 5% by weight to about 25% by weight. Preferably, the primary blend has a residual moisture content after cooking from about 9% by weight to about 20% by weight, for example, about 9% to about 10%, about 10% to about 11%, about 11% to about 12%, about 12% to about 13%, about 13% to about 14%, and about 14% to about 15%, about 15% to about 16%, about 16% to about 17%, about 17% to about 18%, about 18% to about 19%, and about 19% to about 20%. Generally, the primary blend may be cooked at a temperature of about 230° F. to about 250° F., for example, about 230° F. to about 235° F., about 235° F. to about 240° F., about 240° F. to about 245° F., and about 245° F. to about 250° F.

A secondary blend may be added to the primary blend after cooking is completed. The secondary blend may contain one or more components of the semi-solid dosage form. In some embodiments, the secondary blend includes chlorpheniramine maleate, phenylephrine hydrochloride, and hydrolyzed gelatin. In other embodiments, the secondary blend includes calcium carbonate, simethicone, famotidine, or omeprazole, and hydrolyzed gelatin. Water may be added to the secondary blend to dissolve water-soluble components and/or form a homogenous mixture. Other components may be added to the secondary blend including, for example, glycerin, flavorants and colorants. Alternatively, an additional blend may be prepared containing glycerin, flavorants and colorants. An acid solution may further be prepared containing citric acid to obtain the desired pH of the final blend. The final blend may be obtained by combining the primary blend, secondary blend, additional blend and citric acid in any order.

The final blend may be further processed as needed prior to preparation of the semi-solid dosage form. For example, the final blend may be transferred to a depositor hopper having a jacket to maintain a temperature of from about 180° F. to about 210° F., preferably about 190° to about 200° F. After a suitable amount of time, the final blend may be dispensed from the depositor hopper to product the semi-solid chewable dosage form of the invention.

The semi-solid chewable dosage form may be obtained by depositing the final blend into pre-formed plastic molds using conventional techniques. Preferably, the plastic molds are blister packs having multiple cavities that provide for unit dose packaging of the semi-solid dosage form without having to transfer the dosage form from a mold to a separate container. The dosage form solidifies in the plastic molds which serve as the final packaging. As the temperature of the dosage form cools, the dosage form takes its final shape in the cavities of the blister pack. The blister pack is preferably sealed, for example, using foil. One or more blister packs may be packaged in containers. Alternatively, the dosage forms may be prepared in molds and transferred to other suitable containers.

Advantageously, a pre-determined amount of the final blend, for example based on weight, is dispensed into each cavity to form individual pieces. The individual pieces contain the desired amount of the active ingredients as described herein. For example, individual pieces may contain 4 mg chlorpheniramine maleate and 10 mg phenylephrine hydrochloride for an adult dose and 2 mg chlorpheniramine maleate and 5 mg phenylephrine hydrochloride for a pediatric dose.

The following examples further illustrate the invention but, of course, should not be construed as in any way limiting its scope.

### Example 1

This example demonstrates a semi-solid chewable dosage form and its method of preparation in accordance with an embodiment of the invention. A 200 g batch is produced in this example. Each individual piece weighs 5 grams and contains 2 mg of chlorpheniramine maleate and 5 mg of phenylephrine hydrochloride.

| | Formula | Batch |
|---|---|---|
| Ingredient | % by weight | 200 g |
| Sugar (powder) | 40.17 | 80.34 |
| Corn Syrup (dry) | 41.43 | 82.86 |
| Sodium Citrate (powder) | 0.39 | 0.78 |
| USP Citrus Pectin 200 (high methoxy) | 1.97 | 3.94 |
| Residual Water | 11.13 | 22.26 |
| Chlorpheniramine Maleate | 0.04 | 0.08 |
| Phenylephrine Hydrochloride | 0.10 | 0.20 |
| Hydrolyzed Gelatin | 1.48 | 2.96 |
| Artificial sweetener (Sucralose) | 0.30 | 0.6 |
| Dodecalactone (1% in PG sol) | 0.01 | 0.02 |
| FD&C Yellow #6 | 0.01 | 0.02 |
| Glycerin USP | 1.97 | 3.94 |
| Orange Flavor FFS (211P52) | 0.20 | 0.40 |
| Menthol | 0.05 | 0.1 |
| Citric Acid (powder) | 0.75 | 1.5 |

A primary blend is prepared that contains sugar, corn syrup, sodium citrate, pectin and water. The primary blend is cooked to produce a residual moisture content of about 11% by weight. A secondary blend is prepared that contains chlorpheniramine maleate, phenylephrine hydrochloride, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 2

This example demonstrates a semi-solid chewable dosage form and its method of preparation in accordance with an embodiment of the invention. A 200 g batch is produced in this example. Each individual piece weighs 5 grams and contains 2 mg of chlorpheniramine maleate and 5 mg of phenylephrine hydrochloride.

| | Formula | Batch |
|---|---|---|
| Ingredient | % by weight | 200 g |
| Sugar (powder) | 42.75 | 85.50 |
| Hydrogenated Starch Hydrolysate (HSH) (dry) | 38.77 | 77.54 |
| Sodium Citrate (powder) | 0.35 | 0.70 |
| USP Citrus Pectin 200 (high methoxy) | 1.99 | 3.98 |
| Residual Water | 11.20 | 22.40 |
| Chlorpheniramine Maleate | 0.04 | 0.08 |
| Phenylephrine Hydrochloride | 0.10 | 0.20 |
| Hydrolyzed Gelatin | 1.49 | 2.98 |
| Artificial sweetener (Sucralose) | 0.30 | 0.6 |
| Dodecalactone (1% in PG sol) | 0.01 | 0.02 |
| FD&C Yellow #6 | 0.01 | 0.02 |
| Glycerin USP | 1.99 | 3.98 |
| Orange Flavor FFS (211P52) | 0.20 | 0.40 |
| Menthol | 0.05 | 0.10 |
| Citric Acid (powder) | 0.75 | 1.50 |

A primary blend is prepared that contains sugar, hydrogenated starch hydrolysate, sodium citrate, pectin and water. The primary blend is cooked to produce a residual moisture content of about 11% by weight. A secondary blend is prepared that contains chlorpheniramine maleate, phenylephrine hydrochloride, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 3

This example demonstrates a semi-solid chewable dosage form and its method of preparation in accordance with an embodiment of the invention. A 200 g batch is produced in this example. Each individual piece weighs 5 grams and contains 4 mg of chlorpheniramine maleate and 10 mg of phenylephrine hydrochloride.

| | Formula | Batch |
|---|---|---|
| Ingredient | % by weight | 200 g |
| Sugar (powder) | 40.12 | 80.24 |
| Corn Syrup (dry) | 41.37 | 82.74 |
| Sodium Citrate (powder) | 0.39 | 0.78 |
| USP Citrus Pectin 200 (high methoxy) | 1.97 | 3.94 |
| Residual Water | 11.10 | 22.20 |
| Chlorpheniramine Maleate | 0.08 | 0.16 |
| Phenylephrine Hydrochloride | 0.20 | 0.4 |
| Hydrolyzed Gelatin | 1.48 | 2.96 |
| Artificial sweetener (Sucralose) | 0.30 | 0.6 |
| Dodecalactone (1% in PG sol) | 0.01 | 0.02 |
| FD&C Red #40 | 0.01 | 0.02 |
| Glycerin USP | 1.97 | 3.94 |
| Cherry Flavor FFS (223G12) | 0.20 | 0.40 |
| Menthol | 0.05 | 0.1 |
| Citric Acid (powder) | 0.75 | 1.5 |

A primary blend is prepared that contains sugar, corn syrup, sodium citrate, pectin and water. The primary blend is cooked to produce a residual moisture content of about 11% by weight. A secondary blend is prepared that contains chlorpheniramine maleate, phenylephrine hydrochloride, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 4

This example demonstrates a semi-solid chewable dosage form and its method of preparation in accordance with an embodiment of the invention. A 200 g batch is produced in this example. Each individual piece weighs 5 grams and contains 4 mg of chlorpheniramine maleate and 10 mg of phenylephrine hydrochloride

| | Formula | Batch |
|---|---|---|
| Ingredient | % by weight | 200 g |
| Sugar (powder) | 42.69 | 85.38 |
| Hydrogenated Starch Hydrolysate (HSH) (dry) | 38.72 | 77.44 |
| Sodium Citrate (powder) | 0.35 | 0.70 |
| USP Citrus Pectin 200 (high methoxy) | 1.99 | 3.98 |
| Residual Water | 11.17 | 22.34 |
| Chlorpheniramine Maleate | 0.08 | 0.16 |
| Phenylephrine Hydrochloride | 0.20 | 0.40 |
| Hydrolyzed Gelatin | 1.49 | 2.98 |
| Artificial sweetener (Sucralose) | 0.30 | 0.6 |
| Dodecalactone (1% in PG sol) | 0.01 | 0.02 |
| FD&C Red #40 | 0.01 | 0.02 |
| Glycerin USP | 1.99 | 3.98 |
| Cherry Flavor FFS (223G12) | 0.20 | 0.40 |
| Menthol | 0.05 | 0.10 |
| Citric Acid (powder) | 0.75 | 1.50 |

A primary blend is prepared that contains sugar, hydrogenated starch hydrolysate, sodium citrate, pectin and water. The primary blend is cooked to produce a residual moisture content of about 11% by weight. A secondary blend is prepared that contains chlorpheniramine maleate, phenylephrine hydrochloride, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 5

This example demonstrates a semi-solid chewable dosage form and its method of preparation in accordance with an embodiment of the invention. A 200 g batch is produced in this example. Each individual piece weighs 5 grams and contains 200 mg of guaifenesin.

| | Formula | Batch |
|---|---|---|
| Ingredient | % by weight | 200 gr |
| Sugar (powder) | 34.00 | 68.00 |
| Hydrogenated Starch Hydrolysate (HSH) (dry) | 38.00 | 76.00 |
| Sodium Citrate (powder) | 0.40 | 0.80 |
| USP Citrus Pectin 200 (high methoxy) | 2.99 | 5.98 |
| Residual Water | 13.79 | 27.58 |
| Guaifenesin (USP) powder | 4.00 | 8.00 |
| Hydrolyzed Gelatin | 3.00 | 6.00 |
| Artificial sweetener (Sucralose) | 0.50 | 1.00 |
| Dodecalactone (1% in PG sol) | 0.02 | 0.04 |
| FD&C Red #40 | 0.01 | 0.02 |
| Glycerin USP | 1.99 | 3.98 |
| Cherry Flavor FFS (223G12) | 0.20 | 0.40 |
| Menthol | 0.10 | 0.20 |
| Citric Acid (powder) | 1.00 | 2.00 |

A primary blend is prepared that contains sugar, hydrogenated starch hydrolysate, sodium citrate, pectin and water. The primary blend is cooked to produce a residual moisture content of about 14% by weight. A secondary blend is prepared that contains guaifenesin, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 6

This example demonstrates a semi-solid chewable dosage form and its method of preparation in accordance with an embodiment of the invention. A 200 g batch is produced in this example. Each individual piece weighs 5 grams and contains 10 mg of dextromethorphan HBr and 5 mg of phenylephrine hydrochloride.

| | Formula | Batch |
|---|---|---|
| Ingredient | % by weight | 200 g |
| Sugar (powder) | 40.10 | 80.20 |
| Corn Syrup (dry) | 41.37 | 82.74 |
| Sodium Citrate (powder) | 0.39 | 0.78 |
| USP Citrus Pectin 200 (high methoxy) | 1.97 | 3.94 |
| Residual Water | 11.10 | 22.20 |
| Dextromethorphan Hydrobromide | 0.20 | 0.40 |
| Phenylephrine Hydrochloride | 0.10 | 0.20 |
| Hydrolyzed Gelatin | 1.48 | 2.96 |
| Artificial sweetener (Sucralose) | 0.30 | 0.6 |
| Dodecalactone (1% in PG sol) | 0.01 | 0.02 |
| FD&C Red #40 | 0.01 | 0.02 |
| Glycerin USP | 1.97 | 3.94 |
| Cherry Flavor FFS (223G12) | 0.20 | 0.40 |
| Menthol | 0.05 | 0.1 |
| Citric Acid (powder) | 0.75 | 1.5 |

A primary blend is prepared that contains sugar, corn syrup, sodium citrate, pectin and water. The primary blend is cooked to produce a residual moisture content of about 11% by weight. A secondary blend is prepared that contains dextromethorphan hydrobromide, phenylephrine hydrochloride, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 7

This example demonstrates a semi-solid chewable dosage form and its method of preparation in accordance with an embodiment of the invention. A 200 g batch is produced in this example. Each individual piece weighs 5 grams and contains 10 mg of dextromethorphan hydrobromide.

| | Formula | Batch |
|---|---|---|
| Ingredient | % by weight | 200 g |
| Sugar (powder) | 40.20 | 80.40 |
| Corn Syrup (dry) | 41.37 | 82.74 |
| Sodium Citrate (powder) | 0.39 | 0.78 |
| USP Citrus Pectin 200 (high methoxy) | 1.97 | 3.94 |
| Residual Water | 11.10 | 22.20 |
| Dextromethorphan Hydrobromide | 0.20 | 0.40 |
| Hydrolyzed Gelatin | 1.48 | 2.96 |
| Artificial sweetener (Sucralose) | 0.30 | 0.60 |
| Dodecalactone (1% in PG sol) | 0.01 | 0.02 |
| FD&C Red #40 | 0.01 | 0.02 |
| Glycerin USP | 1.97 | 3.94 |
| Cherry Flavor FFS (223G12) | 0.20 | 0.40 |
| Menthol | 0.05 | 0.10 |
| Citric Acid (powder) | 0.75 | 1.50 |

A primary blend is prepared that contains sugar, corn syrup, sodium citrate, pectin and water. The primary blend is cooked to produce a residual moisture content of about 11% by weight. A secondary blend is prepared that contains dextromethorphan hydrobromide, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 8

This example demonstrates a semi-solid chewable dosage form and its method of preparation in accordance with an embodiment of the invention. A 200 g batch is produced in this example. Each individual piece weighs 5 grams and contains 10 mg of loratadine.

| | Formula | Batch |
|---|---|---|
| Ingredient | % by weight | 200 g |
| Sugar (powder) | 40.20 | 80.40 |
| Corn Syrup (dry) | 41.37 | 82.74 |
| Sodium Citrate (powder) | 0.39 | 0.78 |
| USP Citrus Pectin 200 (high methoxy) | 1.97 | 3.94 |
| Residual Water | 11.10 | 22.20 |
| Loratadine | 0.20 | 0.40 |
| Hydrolyzed Gelatin | 1.48 | 2.96 |
| Artificial sweetener (Sucralose) | 0.30 | 0.60 |
| Dodecalactone (1% in PG sol) | 0.01 | 0.02 |
| FD&C Red #40 | 0.01 | 0.02 |
| Glycerin USP | 1.97 | 3.94 |
| Cherry Flavor FFS (223G12) | 0.20 | 0.40 |
| Menthol | 0.05 | 0.10 |
| Citric Acid (powder) | 0.75 | 1.5 |

A primary blend is prepared that contains sugar, corn syrup, sodium citrate, pectin and water. The primary blend is cooked to produce a residual moisture content of about 11% by weight. A secondary blend is prepared that contains loratadine, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 9

This example demonstrates a semi-solid chewable dosage form and its method of preparation in accordance with an embodiment of the invention. A 200 g batch is produced in this example. Each individual piece weighs 5 grams and contains 750 mg of calcium carbonate and 80 mg of simethicone.

| | Formula | Batch |
|---|---|---|
| Ingredient | % by weight | 200 g |
| Sugar (powder) | 24.45 | 48.90 |
| Corn Syrup (dry) | 32.20 | 64.40 |
| Amidated Pectin 100 (USP) | 5.00 | 10.00 |
| Residual Water | 18.75 | 37.50 |
| Precipitated Calcium Carbonate | 15.00 | 30.00 |
| Simethicone (USP) | 1.60 | 3.20 |
| Hydrolyzed Gelatin | 1.48 | 2.96 |
| Artificial sweetener (Sucralose) | 0.20 | 0.40 |
| Dodecalactone (0.5% in PG sol) | 0.01 | 0.02 |
| FD&C Red #40 | 0.01 | 0.02 |
| Glycerin (USP) | 1.00 | 2.00 |
| Cherry Flavor FFS (223G12) | 0.25 | 0.50 |
| Menthol | 0.05 | 0.10 |

A primary blend is prepared that contains sugar, corn syrup, pectin, calcium carbonate and water. The primary blend is cooked to 230° F. to obtain a residual moisture content of about 18%-19% by weight. A secondary blend is prepared that contains simethicone, hydrolyzed gelatin, sucralose and dodecalactone (0.5% in propylene glycol solution). An additional blend is prepared that contains glycerin, colorants and flavorants.

The secondary blend and additional blend are combined with the primary blend to form the final blend. The final blend is mixed thoroughly. The final blend is transferred to a depositor hopper. From the depositor hopper, individual pieces are deposited into pre-formed plastic molds.

### Example 10

This example demonstrates a semi-solid chewable dosage form and its method of preparation in accordance with an embodiment of the invention. A 200 g batch is produced in this example. Each individual piece weighs 5 grams and contains 80 mg of simethicone.

| | Formula | Batch |
|---|---|---|
| Ingredient | % by weight | 200 g |
| Sugar (powder) | 34.45 | 69.10 |
| Corn Syrup (dry) | 37.20 | 74.40 |
| Amidated Pectin 100 (USP) | 5.00 | 10.00 |
| Residual Water | 18.75 | 37.50 |
| Simethicone (USP) | 1.60 | 3.20 |
| Hydrolyzed Gelatin | 1.48 | 2.96 |
| Artificial sweetener (Sucralose) | 0.10 | 0.20 |
| Dodecalactone (0.5% in PG sol) | 0.01 | 0.02 |
| FD&C Red #40 | 0.01 | 0.02 |
| Glycerin (USP) | 1.00 | 2.00 |
| Cherry Flavor FFS (223G12) | 0.25 | 0.50 |
| Menthol | 0.05 | 0.10 |

A primary blend is prepared that contains sugar, corn syrup, pectin, and water. The primary blend is cooked to 230° F. to obtain a residual moisture content of about 18%-19% by weight. A secondary blend is prepared that contains simethicone, hydrolyzed gelatin, sucralose and dodecalactone (0.5% in propylene glycol solution). An additional blend is prepared that contains glycerin, colorants and flavorants.

The secondary blend and additional blend are combined with the primary blend to form the final blend. The final blend is mixed thoroughly. The final blend is transferred to a depositor hopper. From the depositor hopper, individual pieces are deposited into pre-formed plastic molds.

### Example 11

This example demonstrates a semi-solid chewable dosage form and its method of preparation in accordance with an embodiment of the invention. A 200 g batch is produced in this example. Each individual piece weighs 5 grams and contains 200 mg of aluminum hydroxide, 200 mg of magnesium hydroxide, and 20 mg of simethicone.

| | Formula | Batch |
|---|---|---|
| Ingredient | % by weight | 200 g |
| Sugar (powder) | 34.45 | 69.10 |
| Corn Syrup (dry) | 30.28 | 60.56 |
| Amidated Pectin 100 (USP) | 5.00 | 10.00 |
| Residual Water | 18.75 | 37.50 |
| Simethicone (USP) | 0.40 | 0.80 |
| Aluminum Hydroxide (USP) | 4.00 | 8.00 |
| Magnesium Hydroxide (USP) | 4.00 | 8.00 |
| Hydrolyzed Gelatin | 1.50 | 3.00 |
| Artificial sweetener (Sucralose) | 0.20 | 0.40 |
| Dodecalactone (0.5% in PG sol) | 0.01 | 0.02 |
| FD&C Red #40 | 0.01 | 0.02 |
| Glycerin (USP) | 1.00 | 2.00 |
| Cherry Flavor FFS (223G12) | 0.25 | 0.50 |
| Menthol | 0.05 | 0.10 |

A primary blend is prepared that contains sugar, corn syrup, pectin, and water. The primary blend is cooked to 230° F. to obtain a residual moisture content of about 18%-19% by weight. A secondary blend is prepared that contains simethicone, aluminum hydroxide, magnesium hydroxide, hydrolyzed gelatin, sucralose and dodecalactone (0.5% in propylene glycol solution). An additional blend is prepared that contains glycerin, colorants and flavorants.

The secondary blend and additional blend are combined with the primary blend to form the final blend. The final blend is mixed thoroughly. The final blend is transferred to a depositor hopper. From the depositor hopper, individual pieces are deposited into pre-formed plastic molds.

### Example 12

This example demonstrates a semi-solid chewable dosage form and its method of preparation in accordance with an embodiment of the invention. A 200 g batch is produced in this example. Each individual piece weighs 5 grams and contains 800 mg of calcium carbonate, 165 mg of magnesium hydroxide, and 10 mg of famotidine.

| | Formula | Batch |
|---|---|---|
| Ingredient | % by weight | 200 g |
| Sugar (powder) | 25.00 | 50.00 |
| Corn Syrup (dry) | 28.63 | 57.26 |
| Amidated Pectin 100 (USP) | 5.00 | 10.00 |
| Residual Water | 18.75 | 37.50 |
| Famotidine (USP) | 0.20 | 0.40 |
| Calcium Carbonate (USP) | 16.00 | 32.00 |
| Magnesium Hydroxide (USP) | 3.30 | 6.60 |
| Hydrolyzed Gelatin | 1.50 | 3.00 |
| Artificial sweetener (Sucralose) | 0.30 | 0.60 |
| Dodecalactone (0.5% in PG sol) | 0.01 | 0.02 |
| FD&C Red #40 | 0.01 | 0.02 |
| Glycerin (USP) | 1.00 | 2.00 |
| Cherry Flavor FFS (223G12) | 0.25 | 0.50 |
| Menthol | 0.05 | 0.10 |

A primary blend is prepared that contains sugar, corn syrup, pectin, and water. The primary blend is cooked to 230° F. to obtain a residual moisture content of about 18%-19% by weight. A secondary blend is prepared that contains famotidine, calcium carbonate, magnesium hydroxide, hydrolyzed gelatin, sucralose and dodecalactone (0.5% in propylene glycol solution). An additional blend is prepared that contains glycerin, colorants and flavorants.

The secondary blend and additional blend are combined with the primary blend to form the final blend. The final blend is mixed thoroughly. The final blend is transferred to a depositor hopper. From the depositor hopper, individual pieces are deposited into pre-formed plastic molds.

### Example 13

This example demonstrates a semi-solid chewable dosage form and its method of preparation in accordance with an embodiment of the invention. A 200 g batch is produced in this example. Each individual piece weighs 5 grams and contains 10 mg of famotidine.

| | Formula | Batch |
|---|---|---|
| Ingredient | % by weight | 200 g |
| Sugar (powder) | 40.00 | 80.00 |
| Corn Syrup (dry) | 32.93 | 65.86 |
| Amidated Pectin 100 (USP) | 5.00 | 10.00 |
| Residual Water | 18.75 | 37.50 |
| Famotidine (USP) | 0.20 | 0.40 |
| Hydrolyzed Gelatin | 1.50 | 3.00 |
| Artificial sweetener (Sucralose) | 0.30 | 0.60 |
| Dodecalactone (0.5% in PG sol) | 0.01 | 0.02 |
| FD&C Red #40 | 0.01 | 0.02 |
| Glycerin (USP) | 1.00 | 2.00 |
| Cherry Flavor FFS (223G12) | 0.25 | 0.50 |
| Menthol | 0.05 | 0.10 |

A primary blend is prepared that contains sugar, corn syrup, pectin, and water. The primary blend is cooked to 230° F. to obtain a residual moisture content of about 18%-19% by weight. A secondary blend is prepared that contains famotidine, hydrolyzed gelatin, sucralose and dodecalactone (0.5% in propylene glycol solution). An additional blend is prepared that contains glycerin, colorants and flavorants.

The secondary blend and additional blend are combined with the primary blend to form the final blend. The final blend is mixed thoroughly. The final blend is transferred to a depositor hopper. From the depositor hopper, individual pieces are deposited into pre-formed plastic molds.

### Example 14

This example demonstrates a semi-solid chewable dosage form and its method of preparation in accordance with an embodiment of the invention. A 200 g batch is produced in this example. Each individual piece weighs 5 grams and contains 20 mg of omeprazole.

| | Formula | Batch |
|---|---|---|
| Ingredient | % by weight | 200 g |
| Sugar (powder) | 40.00 | 80.00 |
| Corn Syrup (dry) | 32.73 | 65.46 |
| Amidated Pectin 100 (USP) | 5.00 | 10.00 |
| Residual Water | 18.75 | 37.50 |
| Omeprazole (USP) | 0.40 | 0.80 |
| Hydrolyzed Gelatin | 1.50 | 3.00 |
| Artificial sweetener (Sucralose) | 0.30 | 0.60 |
| Dodecalactone (0.5% in PG sol) | 0.01 | 0.02 |
| FD&C Red #40 | 0.01 | 0.02 |
| Glycerin (USP) | 1.00 | 2.00 |
| Cherry Flavor FFS (223G12) | 0.25 | 0.50 |
| Menthol | 0.05 | 0.10 |

A primary blend is prepared that contains sugar, corn syrup, pectin, and water. The primary blend is cooked to 230° F. to obtain a residual moisture content of about 18%-19% by weight. A secondary blend is prepared that contains omeprazole, hydrolyzed gelatin, sucralose and dodecalactone (0.5% in propylene glycol solution). An additional blend is prepared that contains glycerin, colorants and flavorants.

The secondary blend and additional blend are combined with the primary blend to form the final blend. The final blend is mixed thoroughly. The final blend is transferred to a depositor hopper. From the depositor hopper, individual pieces are deposited into pre-formed plastic molds.

### Example 15

This example demonstrates a semi-solid chewable dosage form and its method of preparation in accordance with an embodiment of the invention. A 200 g batch is produced in this example. Each individual piece weighs 5 grams and contains 80 mg of simethicone.

| | Formula | Batch |
|---|---|---|
| Ingredient | % by weight | 200 g |
| Sugar (powder) | 38.51 | 77.02 |
| Hydrogenated Starch Hydrolysate (dry) | 37.20 | 74.40 |
| Sodium Citrate (powder) | 0.40 | 0.80 |
| USP Citrus Pectin 200 (high methoxyl) | 2.99 | 5.98 |
| Residual Water | 13.79 | 27.58 |
| Simethicone (USP) | 1.60 | 3.20 |
| Hydrolyzed Gelatin | 2.00 | 4.00 |
| Artificial sweetener (Sucralose) | 0.20 | 0.40 |
| Dodecalactone (1.0% in PG sol) | 0.01 | 0.02 |
| FD&C Red #40 | 0.01 | 0.02 |
| Glycerin (USP) | 1.99 | 3.98 |
| Cherry Flavor FFS (223G12) | 0.20 | 0.40 |
| Menthol | 0.10 | 0.20 |
| Citric Acid (powder) | 1.00 | 2.00 |

A primary blend is prepared that contains sugar, hydrogenated starch hydrolysate, sodium citrate, pectin, and water. The primary blend is cooked to 230° F. to obtain a residual moisture content of about 13%-14% by weight. A secondary blend is prepared that contains simethicone, hydrolyzed gelatin, sucralose and dodecalactone (1.0% in propylene glycol solution). An additional blend is prepared that contains glycerin, colorants and flavorants.

The secondary blend, additional blend and citric acid are combined with the primary blend to form the final blend. The final blend is mixed thoroughly. The final blend is transferred to a depositor hopper. From the depositor hopper, individual pieces are deposited into pre-formed plastic molds.

### Example 16

This example demonstrates a semi-solid chewable dosage form and its method of preparation in accordance with an embodiment of the invention. A 200 g batch is produced in this example. Each individual piece weighs 5 grams and contains 750 mg of calcium carbonate.

| | Formula | Batch |
|---|---|---|
| Ingredient | % by weight | 200 g |
| Sugar (powder) | 25.05 | 50.10 |
| Hydrogenated Starch Hydrolysate (dry) | 33.30 | 66.40 |
| Amidated Pectin 100 (USP) | 5.00 | 10.00 |
| Residual Water | 18.75 | 37.50 |
| Precipitated Calcium Carbonate | 15.00 | 30.00 |
| Hydrolyzed Gelatin | 1.48 | 2.96 |
| Artificial sweetener (Sucralose) | 0.20 | 0.40 |
| Dodecalactone (0.5% in PG sol) | 0.01 | 0.02 |
| FD&C Red #40 | 0.01 | 0.02 |
| Glycerin (USP) | 1.00 | 2.00 |
| Cherry Flavor FFS (223G12) | 0.25 | 0.50 |
| Menthol | 0.05 | 0.10 |

A primary blend is prepared that contains sugar, hydrogenated starch hydrolysate, pectin, calcium carbonate, and water. The primary blend is cooked to 230° F. to obtain a residual moisture content of about 18%-19% by weight. A secondary blend is prepared that contains hydrolyzed gelatin, sucralose and dodecalactone (0.5% in propylene glycol solution). An additional blend is prepared that contains glycerin, colorants and flavorants.

The secondary blend and additional blend are combined with the primary blend to form the final blend. The final blend is mixed thoroughly. The final blend is transferred to a depositor hopper. From the depositor hopper, individual pieces are deposited into pre-formed plastic molds.

### Example 17

This example demonstrates a semi-solid chewable dosage form and its method of preparation in accordance with an embodiment of the invention. A semi-solid chewable dosage form containing about 4 mg of chlorpheniramine maleate and about 10 mg of phenylephrine hydrochloride is prepared.

| | Formula |
|---|---|
| Ingredient | % by weight |
| Sugar (granular) | 40.44 |
| Hydrogenated Starch Hydrolysate | 49.54 |
| (HSH 3375 75% solids) (dry basis) | |
| Sodium Citrate (powder) | 0.15 |
| Pectin USP L-200 (powder) | 2.02 |
| Chlorpheniramine Maleate | 0.09 |
| Phenylephrine Hydrochloride | 0.22 |
| Hydrolyzed Gelatin (Gelita Peptiplus) | 1.42 |
| Artificial sweetener (Sucralose) | 0.30 |
| Cherry Flavor FFS (223G12) | 0.20 |
| FD&C Red #40 | 0.05 |
| Citric Acid (50/50 solution) (dry basis) | 1.62 |
| Water | 3.95 |

A primary blend is prepared that contains sugar, hydrogenated starch hydrolysate, sodium citrate, pectin and water. The primary blend is cooked to yield a Brix value of about 85°. A secondary blend is prepared that contains chlorpheniramine maleate, phenylephrine hydrochloride, hydrolyzed gelatin, and sucralose. An additional blend is prepared that contains colorants and flavorants. An acid solution is prepared using citric acid.

The secondary blend, additional blend and acid solution are combined with the primary blend to form the final blend. The final blend is mixed thoroughly to yield a Brix value of about 81° to about 83°. The final blend is transferred to a depositor hopper. From the depositor hopper, individual pieces are deposited into pre-formed plastic molds.

### Example 18

This example demonstrates a semi-solid chewable dosage form and its method of preparation in accordance with an embodiment of the invention. A semi-solid chewable dosage form containing about 2 mg of chlorpheniramine maleate and about 5 mg of phenylephrine hydrochloride is prepared.

| | Formula |
|---|---|
| Ingredient | % by weight |
| Sugar (granular) | 39.94 |
| Hydrogenated Starch Hydrolysate | 50.45 |
| (HSH 3375 75% solids) (dry basis) | |
| Sodium Citrate (powder) | 0.16 |
| Pectin USP L-200 (powder) | 2.10 |
| Chlorpheniramine Maleate | 0.04 |
| Phenylephrine Hydrochloride | 0.11 |
| Hydrolyzed Gelatin (Gelita Peptiplus) | 1.47 |
| Artificial sweetener (Sucralose) | 0.11 |
| Orange Flavor FFS (221P52) | 0.29 |
| FD&C Yellow #6 | 0.13 |
| Citric Acid (50/50 solution) (dry basis) | 1.68 |
| Water | 3.52 |

A primary blend is prepared that contains sugar, hydrogenated starch hydrolysate, sodium citrate, pectin and water. The primary blend is cooked to yield a Brix value of about 85°. A secondary blend is prepared that contains chlorpheniramine maleate, phenylephrine hydrochloride, hydrolyzed gelatin, and sucralose. An additional blend is prepared that contains colorants and flavorants. An acid solution is prepared using citric acid.

The secondary blend, additional blend and acid solution are combined with the primary blend to form the final blend. The final blend is mixed thoroughly to yield a Brix value of about 81° to about 83°. The final blend is transferred to a depositor hopper. From the depositor hopper, individual pieces are deposited into pre-formed plastic molds.

### Example 19

This example demonstrates a semi-solid chewable dosage form and its method of preparation in accordance with an embodiment of the invention. A semi-solid chewable dosage form containing about 25 mg of diphenhydramine hydrochloride is prepared.

| | Formula |
|---|---|
| Ingredient | % by weight |
| Sugar (granular) | 40.66 |
| Hydrogenated Starch Hydrolysate | 49.25 |
| (HSH 3375 75% solids) (dry basis) | |
| Sodium Citrate (powder) | 0.16 |
| Pectin USP L-200 (powder) | 2.10 |
| Diphenhydramine Hydrochloride | 0.55 |
| Hydrolyzed Gelatin (Gelita Peptiplus) | 1.43 |
| Artificial sweetener (Sucralose) | 0.23 |
| Grape Flavor FFS (227U64) | 0.27 |
| FD&C Red #40 | 0.16 |
| FD&C Blue #1 | 0.01 |
| Citric Acid (50/50 solution) (dry basis) | 1.57 |
| Water | 3.64 |

A primary blend is prepared that contains sugar, hydrogenated starch hydrolysate, sodium citrate, pectin and water. The primary blend is cooked to yield a Brix value of about 85°. A secondary blend is prepared that contains diphenhydramine hydrochloride, hydrolyzed gelatin, and sucralose. An additional blend is prepared that contains colorants and flavorants. An acid solution is prepared using citric acid.

The secondary blend, additional blend and acid solution are combined with the primary blend to form the final blend. The final blend is mixed thoroughly to yield a Brix value of about 81° to about 83°. The final blend is transferred to a depositor hopper. From the depositor hopper, individual pieces are deposited into pre-formed plastic molds.

### Example 20

This example demonstrates a semi-solid chewable dosage form and its method of preparation in accordance with an embodiment of the invention. A semi-solid chewable dosage form containing about 12.5 mg of diphenhydramine hydrochloride is prepared.

| | Formula |
|---|---|
| Ingredient | % by weight |
| Sugar (granular) | 40.80 |
| Hydrogenated Starch Hydrolysate | 49.42 |
| (HSH 3375 75% solids) (dry basis) | |
| Sodium Citrate (powder) | 0.16 |
| Pectin USP L-200 (powder) | 2.10 |
| Diphenhydramine Hydrochloride | 0.28 |
| Hydrolyzed Gelatin (Gelita Peptiplus) | 1.43 |
| Artificial sweetener (Sucralose) | 0.23 |
| Grape Flavor FFS (227U64) | 0.27 |
| FD&C Red #40 | 0.16 |
| FD&C Blue #1 | 0.01 |
| Citric Acid (50/50 solution) (dry basis) | 1.57 |
| Water | 3.57 |

A primary blend is prepared that contains sugar, hydrogenated starch hydrolysate, sodium citrate, pectin and water. The primary blend is cooked to yield a Brix value of about 85°. A secondary blend is prepared that contains diphenhydramine hydrochloride, hydrolyzed gelatin, and sucralose. An additional blend is prepared that contains colorants and flavorants. An acid solution is prepared using citric acid.

The secondary blend, additional blend and acid solution are combined with the primary blend to form the final blend. The final blend is mixed thoroughly to yield a Brix value of about 81° to about 83°. The final blend is transferred to a depositor hopper. From the depositor hopper, individual pieces are deposited into pre-formed plastic molds.

### Example 21

This example demonstrates a semi-solid chewable dosage form and its method of preparation in accordance with an embodiment of the invention. A semi-solid chewable dosage form containing about 10 mg of loratadine is prepared.

| | Formula |
|---|---|
| Ingredient | % by weight |
| Sugar (granular) | 39.78 |
| Hydrogenated Starch Hydrolysate | 50.25 |
| (HSH 3375 75% solids) (dry basis) | |
| Sodium Citrate (powder) | 0.16 |
| Pectin USP L-200 (powder) | 2.09 |
| Loratadine | 0.22 |
| Glycerin USP | 1.88 |
| Cherry Flavor FFS (223G12) | 0.27 |
| FD&C Red #40 | 0.19 |
| Citric Acid (50/50 solution) (dry basis) | 1.57 |
| Water | 3.59 |

A primary blend is prepared that contains sugar, hydrogenated starch hydrolysate, sodium citrate, pectin and water. The primary blend is cooked to yield a Brix value of about 85°. A secondary blend is prepared that contains loratadine and glycerin. An additional blend is prepared that contains colorants and flavorants. An acid solution is prepared using citric acid.

The secondary blend, additional blend and acid solution are combined with the primary blend to form the final blend. The final blend is mixed thoroughly to yield a Brix value of about 81° to about 83°. The final blend is transferred to a depositor hopper. From the depositor hopper, individual pieces are deposited into pre-formed plastic molds.

### Example 22

This example demonstrates a semi-solid chewable dosage form and its method of preparation in accordance with an embodiment of the invention. A semi-solid chewable dosage form containing about 10 mg of loratadine is prepared.

| | Formula |
|---|---|
| Ingredient | % by weight |
| Sugar (granular) | 39.78 |
| Hydrogenated Starch Hydrolysate | 50.25 |
| (HSH 3375 75% solids) (dry basis) | |
| Sodium Citrate (powder) | 0.16 |
| Pectin USP L-200 (powder) | 2.09 |
| Loratadine | 0.22 |
| Glycerin USP | 1.88 |
| Grape Flavor FFS (227U64) | 0.21 |
| FD&C Red #40 | 0.16 |
| FD&C Blue #1 | 0.01 |
| Citric Acid (50/50 solution) (dry basis) | 1.57 |
| Water | 3.67 |

A primary blend is prepared that contains sugar, hydrogenated starch hydrolysate, sodium citrate, pectin and water. The primary blend is cooked to produce a residual moisture content of about 85% solids. A secondary blend is prepared that contains loratadine and glycerin. An additional blend is prepared that contains colorants and flavorants. An acid solution is prepared using citric acid.

The secondary blend, additional blend and acid solution are combined with the primary blend to form the final blend. The final blend is mixed thoroughly to yield a Brix value of about 81° to about 83°. The final blend is transferred to a depositor hopper. From the depositor hopper, individual pieces are deposited into pre-formed plastic molds.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted y context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A semi-solid chewable dosage form comprising an active pharmaceutical ingredient, a gelling agent, gelatin, sugar, a polyol, and a pH adjusting agent.

2. The semi-solid chewable dosage form of claim 1, wherein the gelling agent is pectin, the gelatin is hydrolyzed gelatin, and the polyol is hydrolyzed starch hydrolysate.

3. The semi-solid dosage form of claim 2, wherein pectin is present in an amount from about 0.5% by weight to about 7% by weight, hydrolyzed gelatin is present in an amount from about 0.5% by weight to about 8% by weight, and hydrolyzed starch hydrolysate is present in an amount from about 40% by weight to about 90% by weight.

4. The semi-solid chewable dosage form of claim 1, wherein the active pharmaceutical ingredient is diphenhydramine hydrochloride.

5. The semi-solid chewable dosage form of claim 4, wherein the gelling agent is pectin.

6. The semi-solid chewable dosage form of claim 4, wherein the gelatin is hydrolyzed gelatin.

7. The semi-solid chewable dosage form of claim 4, wherein the polyol is hydrolyzed starch hydrolysate.

8. The semi-solid dosage form of claim 4, wherein the gelling agent is pectin present in an amount from about 0.5% by weight to about 7% by weight, the gelatin is hydrolyzed gelatin present in an amount from about 0.5% by weight to about 8% by weight, and the polyol is hydrolyzed starch hydrolysate present in an amount from about 40% by weight to about 90% by weight.

9. A semi-solid chewable dosage form comprising:
an active pharmaceutical ingredient;
pectin in an amount from about 0.5% by weight to about 7% by weight;
sugar in an amount from about 40% by weight to about 95% by weight;
hydrolyzed starch hydrolysate in an amount from about 40% by weight to about 90% by weight;
hydrolyzed gelatin in an amount from about 0.5% by weight to about 8% by weight;
sodium citrate in an amount from about 0.1% by weight to about 1% by weight; and
citric acid in an amount from about 0.5% by weight to about 3% by weight;
wherein the water content of the semi-solid dosage form is from about 8% by weight to about 15% by weight.

10. The semi-solid chewable dosage form of claim 9, wherein the active pharmaceutical ingredient is diphenhydramine hydrochloride.

11. The semi-solid dosage form of claim 9, wherein pectin is present in an amount from about 1% by weight to about 5% by weight, hydrolyzed gelatin is present in an amount from about 1% by weight to about 5% by weight, and the hydrolyzed starch hydrolysate present in an amount from about 40% by weight to about 60% by weight.

12. A semi-solid chewable dosage form comprising an active pharmaceutical ingredient, a gelling agent, sugar, a polyol, glycerin, and a pH adjusting agent.

13. The semi-solid chewable dosage form of claim 12, wherein the gelling agent is pectin and the polyol is hydrolyzed starch hydrolysate.

14. The semi-solid dosage form of claim 13, wherein pectin is present in an amount from about 0.5% by weight to about 7% by weight, hydrolyzed starch hydrolysate present in an amount from about 40% by weight to about 90% by weight, and glycerin is present in an amount from about 0.5% by weight to about 5% by weight.

15. The semi-solid chewable dosage form of claim 12, wherein the active pharmaceutical ingredient is loratadine.

16. The semi-solid chewable dosage form of claim 15, wherein the gelling agent is pectin.

17. The semi-solid chewable dosage form of claim 15, wherein the gelatin is hydrolyzed gelatin.

18. The semi-solid chewable dosage form of claim 15, wherein the polyol is hydrolyzed starch hydrolysate.

19. The semi-solid dosage form of claim 15, wherein the gelling agent is pectin present in an amount from about 0.5% by weight to about 7% by weight, the polyol is hydrolyzed starch hydrolysate present in an amount from about 40% by weight to about 90% by weight, and glycerin is present in an amount from about 0.5% by weight to about 5% by weight.

20. A semi-solid chewable dosage form comprising:
an active pharmaceutical ingredient;
pectin in an amount from about 0.5% by weight to about 7% by weight;
sugar in an amount from about 40% by weight to about 95% by weight;
hydrolyzed starch hydrolysate in an amount from about 40% by weight to about 90% by weight;
glycerin in an amount from about 0.5% by weight to about 5% by weight;
sodium citrate in an amount from about 0.1% by weight to about 1% by weight; and
citric acid in an amount from about 0.5% by weight to about 3% by weight;
wherein the water content of the semi-solid dosage form is from about 8% by weight to about 15% by weight.

21. The semi-solid chewable dosage form of claim 20, wherein the active pharmaceutical ingredient is loratadine.
